(19) United States
(12) Patent Application Publication
(10) Pub. No.: US 2004/0068201 A1
(43) Pub. Date: Apr. 8, 2004

(54) Systems and methods for flow detection and measurement in CSF shunts

(75) Inventor: Tom Saul, El Granada, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: EUNOE, INC., Redwood City, CA

(21) Appl. No.: 10/367,666

(22) Filed: Feb. 14, 2003

Related U.S. Application Data
(60) Provisional application No. 60/357,401, filed on Feb. 15, 2002.

(51) Int. Cl. A61B 5/00

(52) U.S. Cl. 600/561

(57) ABSTRACT

Devices and methods for removing cerebrospinal fluid (CSF) from a CSF space of a patient at relatively constant flow rates for patients having normal intracranial pressures, e.g. patients not suffering from hydrocephalus. The devices and methods provide drainage paths which permit the removal of CSF at relatively low flow rates, usually below 0.2 ml/day, at normal intracranial pressures, e.g. an intracranial pressure between −170 mm of H2O in upright patients and 200 mm of H2O in reclining patients. The removal of CSF at relatively low, constant rates is particularly suitable for treating Alzheimer's disease and other conditions related to the presence of toxic and/or pathogenic substances in the CSF.

### CROSS-REFERENCES TO RELATED APPLICATIONS

This application claims the benefit of prior Provisional Application No. 60/357,401 (Attorney Docket No. 18050-000900), filed on Feb. 15, 2002, the full disclosure of which is incorporated herein by reference.

### BACKGROUND OF THE INVENTION

1. Field of The Invention

The present invention relates generally to medical devices and methods. More particularly, the present invention relates to devices and methods for monitoring flow through implanted devices which remove cerebrospinal fluid (CSF) from the CSF space of a patient to treat Alzheimer's disease and other “normal” CSF pressure diseases.

Alzheimer's disease (AD) is a degenerative brain disorder which is characterized clinically by progressive loss of memory, cognition, reasoning, judgment, and emotional stability and which gradually leads to profound mental deterioration and ultimately death. Alzheimer's disease is the most common cause of progressive mental failure (dementia) in aged humans and is estimated to represent the fourth most common medical cause of death in the United States. Alzheimer's disease has been observed in all races and ethnic groups worldwide and presents a major current and future public health problem. The disease is currently estimated to affect about two to four million individuals in the United States alone and is presently considered to be incurable.

Recently, a promising treatment for Alzheimer's disease has been proposed. The proposed treatment relies on the removal of cerebrospinal fluid (CSF) from the CSF space (which includes the subarachnoid space, the ventricles, the vertebral column, and the brain interstitial space) of a patient suffering from Alzheimer's disease. The treatment is based on the principle that in at least some cases, the characteristic lesions, referred to as senile (or amyloid) plaques and other characteristic lesions in the brain associated with Alzheimer's disease result from the retention of certain toxic substances in the CSF of the patient. A number of suspected pathogenic substances, including toxic, neurotoxic, and pathogenic substances, have been identified to date, including β-amyloid peptide (Aβ-40, Aβ-42, and other β amyloids), MAP-tau, and the like. It is believed that freshly produced CSF has lower levels or is free of these toxic substances. Thus, it is believed that removal of CSF from the patient's CSF space will reduce the concentration of such substances and significantly forestall the onset and/or progression of Alzheimer's disease. This treatment for Alzheimer's disease has been described in Rubenstein (1998) The Lancet, 351:283-285, and Silverberg et al. (2002) Neurology 59:1139-1145.

Hydrocephalus is another condition which is treated by removing CSF from a patient's CSF space, in particular from the cerebral ventricles. Hydrocephalus is characterized by an elevated intracranial pressure, at some time in the course of the disorder, resulting from excessive production or retention of CSF, and the removal of such excess CSF has been found to be a highly effective treatment for the condition. Numerous specific catheters and shunts have been designed and produced for the treatment of hydrocephalus, occult hydrocephalus, and other CSF disorders.

The removal of CSF for the treatment of either Alzheimer's disease or hydrocephalus can be accomplished using a wide variety of apparatus which are capable of collecting CSF in the CSF space, preferably from the intracranial ventricles, and transporting the collected fluid to a location outside of the CSF space. Usually, the location will be an internal body location, such as the venous system or the peritoneal cavity, which is capable of harmlessly receiving the fluid and any toxic substances, but it is also possible to externally dispose of the CSF using a transcutaneous device. An exemplary system 100 for removing CSF from a patient's CSF space is illustrated in FIG. 1 and includes an access component 120, a disposal component 140, and a flow control component 160.

While the system of FIG. 1 in general will be suitable for the treatment of both Alzheimer's disease and hydrocephalus, specific characteristics of the flow control component will be quite different because of the different nature of the two diseases. Treatment of hydrocephalus is best accomplished by controlling the flow rate of CSF from the CSF space to the disposal location in order to maintain intracranial pressure within normal physiological limits. Particularly suitable flow control characteristics for a flow control module in a hydrocephalus treatment system are illustrated in FIG. 2. FIG. 2 is taken from U.S. Pat. No. 4,781,672 which describes a flow control valve of the type used in the commercially available OSVII® valve unit available from Integra Neurosciences, Inc. Plainsboro, N.J. (formerly available from NMT Neurosciences, Inc., Elekta, Cordis). Briefly, the pressure P is the difference in pressure or “differential pressure” between the CSF space and the disposal location. The patent teaches that the control valve establishes an initial flow rate Q₁ of about 0.4 ml/min. when the differential pressure P reaches an initial level P₁ of 80 mm H₂O and increases to a higher flow rate Q₂ of 0.8 ml/min. as the differential pressure increases to a higher value P₂ of 350 mm H₂O. When pressure P is below P₁, there is essentially no flow. At pressures above P₂, the flow is essentially unrestricted. Such valve flow characteristics are particularly suitable for treating hydrocephalus because for pressures below P₁, there is no need to reduce pressure and thus no need to remove CSF. For pressures from P₁ to P₂, a controlled removal of CSF at or near the expected daily production rate is desired to lower intracranial pressure with minimum risks of removal of excessive amounts of CSF which would lead to overdrainage complications such as slit ventricles, subdural fluid collections and delayed proximal obstruction. When intracranial pressure exceeds P₂, unphysiologically-high pressures are present and rapid removal of CSF is necessary to immediately lower intracranial pressure to a safer level.

Treatment of Alzheimer's disease and other “normal pressure” CSF conditions typically requires use of a different type of shunt than the one used to treat hydrocephalus. Such shunts generally provide for the controlled removal of CSF from the patient without excessive removal of the CSF in a manner which would place the patient at risk. Examples of such a device are found in U.S. Pat. Nos. 5,980,480; 6,264,625; and 6,383,159, each of which is assigned to the assignee of the present invention. The full disclosures of each of these three patents are incorporated herein by reference.

Cerebrospinal fluid shunts used to treat hydrocephalus, Alzheimer's disease and other conditions are prone to dysfunction. In one study of pediatric patients with CSF shunts, shunt failure ranged from 25% to 40% within twelve months of surgery, with a 4-5% risk for each year thereafter. (Sainte-Rose C. Mechanical Complications in Shunts in Pediatric Neurosurgery 1991-92:17:2-9.) In a recent study of 1183 pediatric shunt replacements in 839 patients, over 70% of failures were related to over- or under-drainage of CSF from the CSF space. (Tuli S, et al. Risk Factors for Repeated Cerebrospinal Shunt Failures in Pediatric Patients with Hydrocephalus. J. Neurosurg. 92:31-38, 2000.)

Although the efficacy of CSF shunts is dependent on CSF flow through the shunt, currently available shunt systems do not include means for monitoring flow. Conventional techniques for monitoring flow in CSF shunts are generally invasive, time consuming, expensive, and often inconclusive, and some techniques place the patient at risk of damage to the shunt and central nervous system infection which could lead to the development of hydrocephalus in patients shunted for AD or worsen an already existing hydrocephalus. The “gold standard” methods to evaluate shunt function involve injecting radiopaque compounds into a reservoir in the shunt system and filming the compounds as they flow through the shunt.—a process called “shuntography”. Such shuntography can be used to assess the integrity of the shunt, i.e., confirm a continuous flow pathway or attempt to measure the rate of flow through the shunt.

In one shuntography method, a radioisotope solution, Indium 111 DPTA, is introduced into the inflow reservoir of a shunt and passage of the isotope through the shunt is monitored with a Gamma camera. In another method, a cadmium telluride detector is placed over the shunt reservoir and clearance of radioisotope injected into the reservoir is recorded to measure flow. Yet another method involves injecting iodinated contrast material into the shunt reservoir and taking serial computed tomography (CT) scans (typically at 0-4, 24 and 48 hours) to assess the rate of iodine dissipation from the ventricular system. All of these methods are invasive, in that they require injection of a substance into the CSF via the shunt, thus exposing the patient to risk of central nervous system infection and/or an allergic reaction to the injected contrast material. The technique involving serial CT scans also exposes patients to a significant dose of radiation Such a procedure would create particular risks in growing children. Furthermore, despite being the “gold standards,” these methods are can be inconclusive and are expensive, especially the method requiring serial CT scans.

Non-invasive methods for measuring CSF flow do exist, but they are also typically inconclusive, expensive or both. For example, CSF flow in a shunt may be measured by magnetic resonance imaging (MRI). This is a non-invasive procedure, but is costly and typically allows measurement only in the recumbent position. Thus, the shunt can only be tested in one orientation and does not allow the the clinician to assess flow over a range of body postures. Measurement in the recumbent position eliminates the effects of gravity on shunt performance, thereby limiting the utility of MRI measurements for assessing function of CSF shunts that are in place to reduce intracranial pressure (such as those used to treat hydrocephalus).

Conventional radiographs (“X-rays”) of the brain can show the enlargement or collapse of the ventricles due to under- or over-drainage, respectively. However, an ideal shunt flow measurement technique would show under- or over-drainage long before any change in ventricle size on a plain X-ray is detectable. Furthermore, X-rays cannot typically identify specific locations of the CSF shunt malfunction.

One indirect method for measuring CSF shunt flow is to implant a device to monitor intracranial pressure. For example, one such device is the TeleSensor formerly manufactured by Radionics (a division of Tyco Healthcare, LP—see www.radionics.com, the technology is now owned by Integra Neurosciences, Inc.) operates by radio frequency pressure-balanced telemetry, and is queried transcutaneously. A second similar invention is disclosed by patent application (Ser. No. 909485) dated Jul. 20, 2001 entitled “Device and method to measure and communicate body parameters” by Penn et al. assigned to Medtronic, Inc. This invention improves upon the Telesensor in that it measures absolute pressure correctedion for temperature and barometric and stores an 11-minute sample of high-resolution data (every 2 seconds) triggerd by an event marker that stores pre- and posttrigger data samples. The intent of this feature is to capture data before and during symptomatic periods for later review. However, both of these devices require a patent pathway between the intracranial cavity and the sensor and partial blockages are difficult to assess since a hydrostatic pathway might be sufficient to transmit pressures and waveforms. If blockage occurs proximal to the sensor, intracranial pressure cannot be measured. Furthermore, among patients with the “normal pressure” variant of hydrocephalus, or in individuals shunted to improve CSF clearance for other conditions, such a device would not be useful in monitoring shunt function.

Due to the lack of reliable, conclusive, cost-effective, low-risk methods for measuring flow in CSF shunts, shunt failure typically goes undetected until neurologic symptoms return or worsen. In hydrocephalus, undetected shunt dysfunction can lead to permanent neurological damage or death. In Alzheimer's disease, shunt dysfunction may be more difficult to detect, due to slow worsening of symptoms, and thus may go undetected for long periods of time. By the time such shunt dysfunction would be detectable from observation of the return of symptoms, the patient would have regressed significantly.

In summary, use of an implanted shunt for draining CSF for the treatment of both hydrocephalus and Alzheimer's disease, as well as other types of shunts that drain other body fluids, can fail because of malfunction of the drainage shunt. In particular, the valves will usually be constructed to fail in the closed condition (to prevent catastrophic over drainage of the CSF) and it becomes important to monitor shunt operation to make sure that drainage continues. In the case of hydrocephalus shunts, it has been proposed that integrated pressure monitors, either separate from or provided on the shunt which can alert the patient or treating professional that intracranial pressure has become elevated and that the shunt operation is likely compromised. The use of pressure monitoring for patients suffering from Alzheimer's disease and other “normal pressure” conditions would not be adequate since these patients would not be expected to display elevated intracranial pressure even if the shunt failed.

For these reasons, it would be desirable to provide methods and apparatus for monitoring the proper operation of implanted CSF drainage shunts in patients suffering from Alzheimer's disease and other “normal pressure” conditions, including normal pressure hydrocephalus (NPH). In particular, it would be desirable if such methods and systems were able to detect flow through the shunts, and more particularly, the relatively low flow rates and cumulative flows that would be utilized in the treatment of such normal pressure conditions. It would be further desirable if such methods and apparatus were also useful for detecting flow in “high pressure” shunts used for hydrocephalous.

2. Description of Background Art

The treatment of Alzheimer's disease by removing cerebrospinal fluid from the CSF region of the brain is described in U.S. Pat. Nos. 5,980,480; 6,264,625; and 6,383,159, each of which are assigned to the assignee of the present invention. The full disclosures of each of these three patents are incorporated herein by reference. U.S. Pat. No. 5,334,315, describes treatment of various body fluids, including CSF, to remove pathogenic substances. Methods and shunts for treating hydrocephalus are described in U.S. Pat. Nos. 3,889,687; 3,985,140; 3,913,587; 4,375,816; 4,377,169; 4,385,636; 4,432,853; 4,532,932; 4,540,400; 4,551,128; 4,557,721; 4,576,035; 4,595,390; 4,598,579; 4,601,721; 4,627,832; 4,631,051; 4,675,003; 4,676,772; 4,681,559; 4,705,499; 4,714,458; 4,714,459; 4,769,002; 4,776,838; 4,781,672; 4,787,886; 4,850,955; 4,861,331; 4,867,740; 4,931,039; 4,950,232; 5,039,511; 5,069,663; 5,336,166; 5,368,556; 5,385,541; 5,387,188; 5,437,627; 5,458,606; PCT Publication WO 96/28200; European Publication 421558; 798011; and 798012; French Publication 2 705 574; Swedish Publication 8801516; and SU 1297870. A comparison of the pressure-flow performance of a number of commercially available hydrocephalus shunt devices is presented in Czosnyka et al. (1998) Neurosurgery 42: 327-334. A shunt valve having a three-stage pressure response profile is sold under the OSVII® tradename by Integra (Integra Neurosciences, Inc. Plainsboro, N.J. ((formerly available from NMT Neurosciences, Inc., Elekta, and Cordis). Articles discussing pressures and other characteristics of CSF in the CSF space include Condon (1986) J. Comput. Assit. Tomogr. 10:784-792; Condon (1987) J. Comput. Assit. Tomogr. 11:203-207; Chapman (1990) Neurosurgery 26:181-189; Magneas (1976) J. Neurosurgery 44:698-705; Langfitt (1975) Neurosurgery 22:302-320. Apparatus for measuring and transmitting pressure in an implanted hydrocephalus shunt is described in U.S. Pat. No. 5,704,352. While it is suggested that flow and many other parameters might alternatively be measured, no description of how such measurements might be performed is provided. The measurement of flow and other parameters in other implanted devices is described in U.S. Pat. Nos. 5,357,967; 5,598,847; 5,685,989; 5,833,603; 6,021,415; and 6,170,4;88.

### BRIEF SUMMARY OF THE INVENTION

Methods and apparatus according to the present invention are used in conjunction with low flow, continuous protocols for removal of cerebrospinal fluid (CSF) from the CSF space of a patient. The protocols are usually intended for the treatment of Alzheimer's disease and other normal pressure conditions, such as normal pressure hydrocephalus (NPH) or conditions which are caused by or otherwise related to the retention and accumulation of toxic substances in the CSF. Exemplary conditions which result from the accumulation of toxic substances in the patient's brain, include Down's Syndrome, hereditary cerebral hemorrhage with amyloidosis of the Dutch-Type (HCHWA-D), and the like. Other treatable conditions relating to the chronic or acute presence of potentially putative substances include epilepsy, narcolepsy, Parkinson's disease, polyneuropathies, multiple sclerosis, amyotrophic lateral sclerosis (ALS), myasthenia gravis, muscular dystrophy, dystrophy myotonic, other myotonic syndromes, polymyositis, dermatomyositis, brain tumors, Guillain-Barre-Syndrome, and the like.

The devices and methods of the present invention are particularly intended for the treatment of patients having normal (not elevated) intracranial pressures but in some embodiments may also find use in treating patients suffering from hydrocephalus and other elevated pressure conditions. “Normal” intracranial pressures are considered to be below 200 mm H₂O when the patient is reclining and above −170 mm H₂O when the patient is upright (where the pressures are measured relative to the ambient). In contrast, patients suffering from hydrocephalus (excluding normal pressure hydrocephalus) will have constant or periodic elevated intracranial pressures above 200 mm H₂O (when reclining), often attaining levels two or three times the normal level if untreated. Differences in untreated intracranial and ventricular pressures as well as the different treatment end points (the treatment of hydrocephalus requires lowering of elevated pressures while preferred treatments according to present inventions are usually intended to enhance CSF turnover and/or lower concentrations of substances in the CSF) require significantly different treatment devices and methods. In particular, preferred treatments and methods according to present invention rely on relatively low CSF removal rates, usually in the range from 12 ml/day to 360 ml/day, more usually in the range from 20 ml/day to 300 ml/day, and preferably in the range from 40 ml/day to 150 ml/day. Further preferably, CSF removal at such low rates will occur continuously or at least so long as the intracranial and ventricular pressures do not fall below certain minimal levels, e.g. below about −170 mm H₂O. Such safety thresholds correspond generally to the lowest expected ventricular pressure of the patient when upright. The intracranial and ventricular pressures referred to above are defined or measured as “gauge” pressures, i.e. relative to ambient pressure. The intracranial pressure falls below ambient (0 mmH₂O) as a result of the compliant nature of the CSF space and the column of CSF fluid which is created as the patient sits upright or stands. The ability of the flow control module to maintain a relatively constant flow (as defined below) regardless of the variations in the intracranial or ventricular “source” pressure is an important aspect of the present invention.

The CSF removal techniques of the present invention may rely on pressure-compensated removal to achieve the desired constant flow rate, where the generally constant (usually varying by no more than ±75%, preferably no more than ±50%, and more preferably ±20%) removal rate is achieved by providing a pressure-controlled variable resistance path in the flow control module between the CSF space and the disposal site. In contrast, the flow control valves for hydrocephalus treatment, such as those described in U.S. Pat. No. 4,781,672, intentionally provide for significant variation in flow rate as the pressure differential across the flow valve passes through specific control points. Use by the present invention of a generally constant flow rate which is below the normal CSF production rate minimizes the possibility of over removal of the CSF and the risk of occlusion associated with CSF stagnation.

Alternatively, the methods and apparatus of the present invention may rely on volumetric CSF removal where target volumes of CSF are removed during predetermined time periods not necessarily being driven by intracranial pressure. Such volumetric removal protocols are described in detail in co-pending application Ser. No. 10/224,046, (Attorney Docket No. 18050-001000US), the full disclosure of which is incorporated herein by reference.

Thus, in a first aspect, methods according to the present invention comprise monitoring cerebral spinal fluid (CSF) flow in a normal pressure patient having an implanted CSF shunt. The methods comprise externally receiving an output signal from a flow sensor in series or parallel with a flow lumen of the implanted CSF drainage shunt, where the signal is representative of CSF flow through the flow lumen. By “externally receiving,” it is meant that the output signal is received by a signal receiver or other device which is located outside of the patient's body. It will be possible, of course, for intermediate transceivers or other repeater devices to be implanted together with the shunt in order to enhance any signal which is being transmitted externally as required by the invention.

In certain embodiments, an interrogation signal will be transmitted to the flow sensor prior to receiving the output signal. Usually, such transmitting is also performed externally, and the transmission signal will also provide power to the flow sensor and/or associated circuitry. Most usually, the transmitted power will activate the flow sensor and provide power to permit flow detection and generation of the output signal.

Exemplary flow sensors include thermal devices, dye release devices, differential pressure measuring devices, turbine meters, angular momentum measuring devices, positive displacement measuring devices, accumulators (which accumulate and track volumes of CSF flow over time), and the like. A presently preferred flow sensor comprises a thermal device which includes a heat generation source and temperature measuring device or sensor located at a known distance from the heat source. The flow signal may then be an inverse function of temperature based on a thermal dilution heat transfer model. Alternatively, the flow signal may be an inverse function of temperature based on a thermal diffusion heat transfer model.

In all of the above embodiments, transmission of the interrogation signal to the flow sensor may comprise directing radiofrequency energy to an antenna coupled to the fluid sensor or circuitry associated with fluid sensor. The radiofrequency energy will provide energy and/or information to the flow sensor, typically providing at least energy, and more usually providing both energy and a signal to initiate flow measurement. The flow measurement will usually be in the range from about 12 ml/day to 360 ml/day, usually from 20 ml/day, to 300 ml/day. Preferably, the signal produced will allow determination of whether there is a flow through the flow lumen at or above a predetermined threshold value, typically at least as low as 12 ml/day. While the flow sensor will usually measure flow rate, it also possible that the flow sensor will monitor cumulative flow over a predetermined time period or periods. For example, flow can be collected in an accumulator over a time period of minutes, hours, or even longer, and a determination then made whether such cumulative flow corresponds to a minimum daily or other flow rate, such as 12 ml/day.

Flow measurements according to the present invention may be performed “instantaneously” or as an average over time. The exemplary thermal measurements (described in detail below) will generally be considered instantaneous since they represent flow over a short period time on the order of seconds. Measured “instantaneous” values may vary over time, and in the case of hydrocephalus (other than normal pressure hydrocephalus), the instantaneous flow values will often if not usually be zero. Average values may be obtained by summing (integrating) the instantaneous values, either electronically or mechanically. The former may be accomplished using hardware or software (either as part of the internal or external system components) which mathematically integrates the instantaneous values over a predetermined time. The latter may be accomplished using an accumulator volume which physically collects CSF and permits periodic or continuous measurement. Combining both approaches, hardware or software can be provided to track and record the CSF flows over time to provide a detailed record of shunt operation.

In addition to radiofrequency energy, other forms of energy, including ultrasonic energy, optical energy, and the like may also be transmitted from an external source to the flow sensor and/or an antenna or other receiver or circuitry associated with the flow sensor. Similarly, the flow sensor and/or other antennas or transmissive components associated with the flow sensor may transmit radiofrequency energy, ultrasonic energy, optical energy, or the like, in order to provide the output signal which is externally received according to the methods of the present invention.

In a second aspect, apparatus according to the present invention for draining CSF comprise an implantable drainage catheter and a flow sensor. The implantable drainage catheter has one end adapted for implantation in a subarachnoid space (SAS) such as from one of the ventricles of the brain, a drainage end adapted for implantation in a drainage space, and flow lumen therebetween. The flow sensor is coupled to sense flow through the flow lumen of the drainage catheter and adapted to transmit a signal representative of flow through the flow lumen, where the sensor is capable of detecting flows at least as slow as 12 ml/day, either as a rate or as an accumulation. Usually, the implantable drainage catheter will include a first conduit implantable in the SAS, a second conduit implantable in the drainage space, and flow control valve assembly therebetween. Typically, the flow sensor will be disposed in or on the flow control valve assembly, although this is not necessary. The flow control valve assembly will usually be configured to allow flows in the ranges and at the ICP's set forth above.

The flow sensor may comprise sensors capable of measuring any of the parameters set forth above, including temperature, differential pressure, dye dilution, the mechanical effects of flow, e.g., as measured by a turbine meter, an angular momentum measuring device, a positive displacement measuring device, an accumulator, or the like, and similar devices. A preferred apparatus will comprise a heater and a temperature detector, where the temperature detector is spaced-apart downstream from the heater, i.e., in a direction toward the drainage end of the catheter, so that the flow signal produced is an inverse function of the temperature measured by the temperature detector based on a thermal dilution heat transfer model, or alternatively a thermal diffusion heat transfer model such as measureing the time for the heater pulse to traverse the distance to the sensor. Usually, the flow sensor will comprise an antenna and associated circuitry for receiving externally generated signals, transmitting signals externally, receiving power, and the like. Most commonly, the antenna will be capable of all three of these functions, i.e., receiving power, receiving signals (such as measurement initiation signals), and transmitting flow measurement data externally back to a user.

In a third aspect, systems according to the present invention for draining CSF and monitoring such drainage comprise an implantable drainage catheter having a flow sensor adapted to detect a flow corresponding to a flow rate at least as low 12 ml/day. The systems will further comprise an external power supply having an antenna adapted to externally deliver energy and optionally signals to the flow sensor when the flow sensor is implanted. The system will still further comprise an external receiver adapted to receive signals from the flow sensor representative of flow through the drainage catheter when the drainage catheter is implanted.

### BRIEF DESCRIPTION OF THE DRAWINGS

FIG. 1 is a schematic illustration showing the components and placement of a conventional system for removing cerebrospinal fluid from a CSF space of the brain.

FIG. 1A is a schematic illustration of the central nervous system, showing the CSF spaces of the brain and spinal cord.

FIG. 2 illustrates the pressure-flow relationship of a conventional flow valve used in systems such as those shown in FIG. 1 for treating hydrocephalus.

FIG. 3 illustrates the pressure-flow relationship of a flow valve used in systems such as those shown in FIG. 1 for treating Alzheimer's disease.

FIG. 4 illustrates one embodiment of an apparatus for measuring flow in a CSF shunt according to the present invention.

FIG. 5 is a schematic illustration of a system according to the present invention for measuring flow in a CSF shunt.

FIGS. 6A-6D illustrate various relationships between temperature and flow, as measured with one embodiment of an apparatus for measuring flow in a CSF-shunt.

FIG. 7 is a schematic illustration of the components of an implantable device constructed in accordance with the principles of the present invention.

FIG. 8 is a schematic illustration of the components of an external receiving device constructed in accordance with the principles of the present invention.

FIG. 9 is a schematic illustration of an alternative implantable device constructed in accordance with the principles of the present invention.

FIG. 10 is a graft showing a representative temperature response curve for the implantable device of FIG. 9.

### DETAILED DESCRIPTION OF THE INVENTION

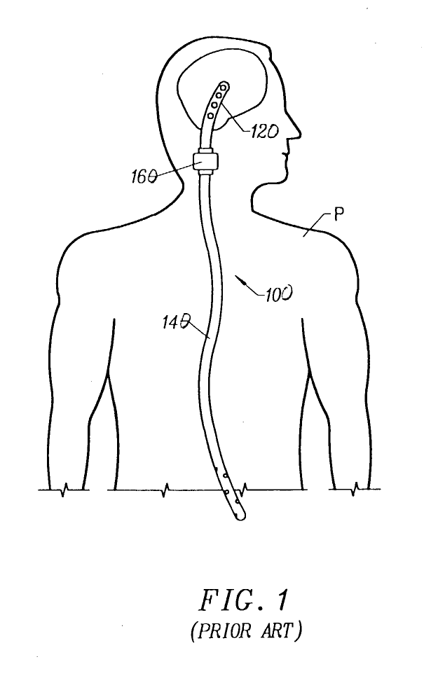

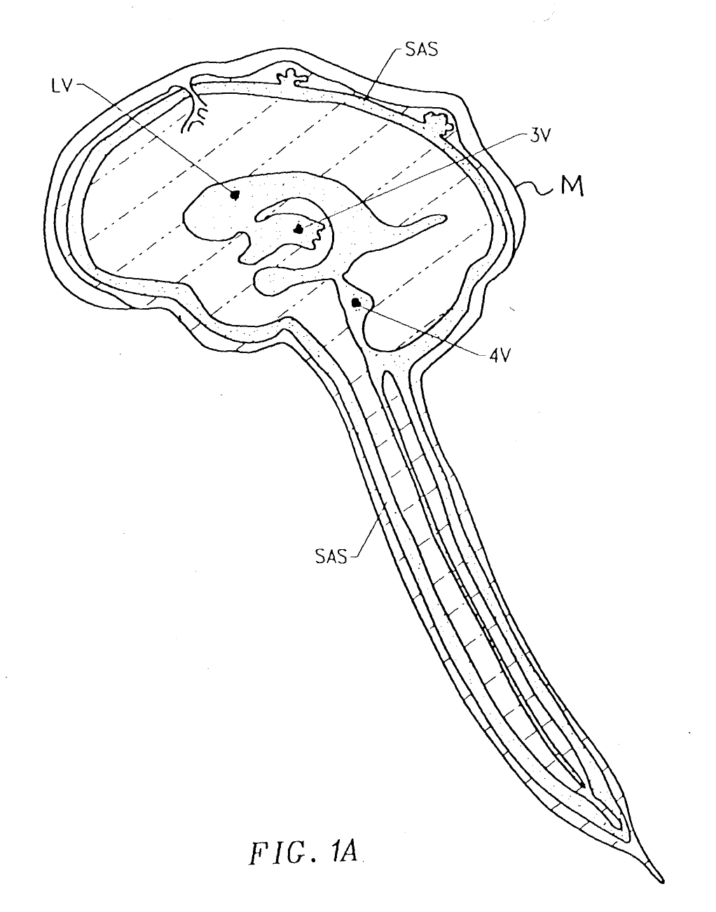

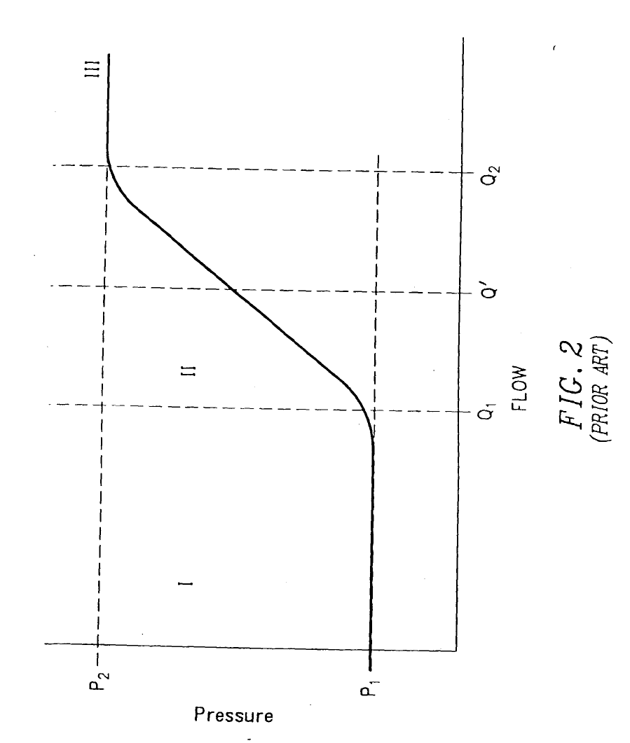

The brain and spinal cord are bathed in cerebrospinal fluid (CSF) and encased within the cranium and vertebral column inside a thin membrane known as the meninges (FIG. 1A). The space within the meninges includes the subarachnoid space SAS, the ventricles (including the lateral ventricle LV, third ventricle 3V, and fourth ventricle 4V), the vertebral column, and the brain interstitial spaces, and is referred to herein as the “CSF space.” The volume of the brain intracranial spaces is on average Iabout 1700 ml. The volume of the brain is approximately 1400 ml, and the volume of the intracranial blood is approximately 150 ml. The remaining 150 ml is filled with CSF (this volume may vary within 60 ml to 290 ml). The CSF circulates within the CSF space. CSF is formed principally by the choroid plexuses, which secrete about 80% of the total volume of the CSF. The sources of the remainder are the vasculature of the subependymal regions, and the pia matter. The total volume of the CSF is renewed several times per day, so that about 500 ml are produced every 24 hours (equivalent to about 20 ml/hr or 0.35 ml/min.) in healthy adults. The production rate is much lower in the old and the very young.

The cerebrospinal fluid is absorbed through the arachnoid villi, located principally over the superior surfaces of the cerebral hemispheres. Some villi also exist at the base of the brain and along the roots of the spinal nerves. The absorptive processes include bulk transport of large molecules and as well as diffusion across porous membranes of small molecules. The production and absorption of CSF are well described in the medical literature. See, e.g., Adams et al. (1989) “Principles of Neurology,” pp. 501-502.

While CSF is naturally absorbed and removed from circulation, as just described, it is presently believed that certain toxic substances which may be present in the CSF, such as those associated with Alzheimer's disease, may accumulate or persist to an extent which can cause Alzheimer's disease or other disorders. Such substances are either produced in excess and/or are removed at a rate slower than their production rate so that they accumulate and increase in toxicity and/or reach a threshold concentration in which they become toxic in the brain or elsewhere within CSF space.

The present invention is particularly directed at devices and methods for detecting low, usually continuous CSF flows in drainage shunts under conditions of “normal pressure,” including but not limited to the removal of toxic substances from the CSF in order to treat, inhibit, or ameliorate conditions associated with such toxic materials. In particular, the present invention is directed at reducing the concentration of such substances in CSF by removing portions of the CSF from the CSF space. Such removal is believed to either enhance production of the CSF and/or reduce the natural absorption of the CSF so that the total volume of CSF in the CSF space is not reduced below a safe level. Since, the rates at which the CSF is removed are generally quite low (when compared to the rates of removal for treatment of the hydrocephalus). The present invention is particularly directed at detecting and confirming such low flow rates. The present invention, however, is not always limited to detection of flow and low pressure and/or low flow conditions, and certain of the detector systems may also find use in monitoring flow through pressure and/or high flow shunts, such as those used for treating hydrocephalus.

By removing CSF from the CSF space, the toxic substances present in the removed CSF will thus be removed from the CSF space and will not be available for absorption or recirculation. So long as the rate of removal exceeds the rate of production of such substances, the concentration of such substances can be reduced. Usually, the removed CSF will be directed to a natural disposal site within the patient's body which can tolerate the toxic substance. Suitable sites, particularly for those substances associated with Alzheimer's disease, include the venous system, peritoneal cavity, the pleural cavity, and the like. In the event that a toxic substance would be deleterious if transferred within the patient's body, or for any other reason, it is also possible to remove the CSF from the patient's body, e.g. using a transcutaneous catheter and external collection bag or other receptacle. It will generally be preferable to maintain the entire system subcutaneously for patient convenience and to reduce the risk of infection.

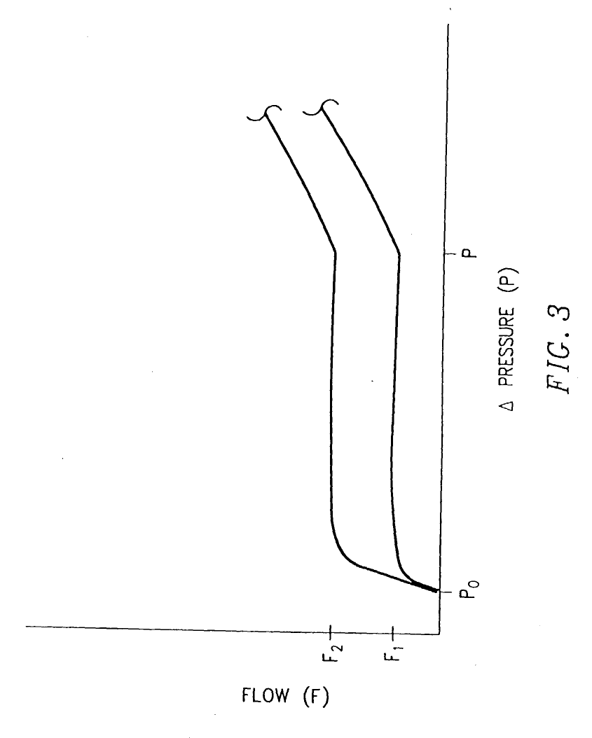

Referring to FIG. 3, the devices and methods of present inventions are usually, but not always, intended to maintain a relatively low constant flow rate of CSF from the CSF space at normal intracranial pressures P (e.g. −170 mm H₂O to 200 mm H₂O relative to ambient). For safety, the devices and methods will be configured to remove little or no CSF at intracranial pressures below a threshold value P₀. At intracranial pressures above P₀, the CSF flow rate F will usually be between a lower value F₁ and an upper value F₂, with particular ranges set forth above. Usually, the flow rate F will be at a relatively constant level, with the rate preferably being pressure-corrected so that it does not vary by more than 75%, preferably by no more than ±50%, and more preferably by no more than ±20% for intracranial pressures within the expected ranges. As observed in FIG. 3, it is desirable that the flow rate F be constant at least over the range P₀ to P₁, and more preferable that the flow rate remains constant for even higher differential pressures since the present invention is not intended to treat excessive intracranial pressure, but rather to remove the CSF at a relatively low, constant rate regardless of the differential pressure (so long as P is above the threshold P₀).

One exemplary apparatus for removing CSF from a CSF space to treat conditions such as Alzheimer's disease is described in U.S. Pat. Nos. 6,264,625 and 6,383,159, both of which have been incorporated herein by reference. In embodiments of the present invention which incorporate a CSF shunt, the shunt may comprise a shunt as described in either of these United States Patents or in any other suitable CSF shunt.

Successful use of CSF shunts is dependent on their ability to control the flow of CSF within the shunt to maintain a normal intracranial pressure. Although the efficacy of CSF shunts is dependent on CSF flow through the shunt, currently available shunt systems do not include means for monitoring flow. Conventional techniques for monitoring flow in CSF shunts, such as shuntograms, MRI scans, serial CT scans, conventional brain imaging and the like, are generally invasive, time consuming, expensive, and often inconclusive, and some techniques place the patient at risk of central nervous system infection. Systems and methods of the present invention provide a non-invasive means for monitoring flow to improve the functionality of CSF shunts for treatment of hydrocephalus, Alzheimer's and other conditions.

Generally, an apparatus for monitoring flow in a CSF shunt according to various aspects of the present invention includes a flow sensor configured to sense flow of CSF through a shunt and transmit a signal representative of that flow. Preferably, such an apparatus is configured to either include an implantable CSF drainage catheter or shunt or to be attachable to a CSF drainage catheter or shunt. Since various embodiments of apparatus according to the present invention will be suitable for use in CSF shunts to treat hydrocephalus, Alzheimer's and other conditions, various embodiments will be designed to operate at flow rates ranging from about 12 ml/day to about 1200 ml/day. Additionally, apparatus according to the present invention will preferably be implantable under a patient's skin. Many suitable embodiments for such an apparatus are contemplated within the scope of the present invention, and the following descriptions of specific embodiments are provided for exemplary purposes only.

Referring to FIG. 4, one embodiment of a flow monitoring apparatus 400 includes a sensor 402, a flow path 406 and a transmitter/receiver 408. Sensor 402 may include a temperature detector 404 and a heater (not shown). Also illustrated in FIG. 4 is a section of the flow lumen 412 of a CSF drainage catheter into which flow monitoring apparatus 400 is incorporated. (For the purpose of this specification, “CSF drainage catheter” and “CSF shunt” are synonymous.) In various alternative embodiments, flow monitoring apparatus 400 may include a CSF drainage catheter or may be a separate apparatus configured for attachment to or incorporation with a CSF drainage catheter.

According to various aspects of the present invention, CSF drainage catheter may comprise a first conduit implantable in the subarachnoid space, a second conduit implantable in a drainage space and a flow control valve positioned between the two (not shown in FIG. 4). In various embodiments of the present invention, flow monitoring apparatus 400 may be positioned on flow control valve or at some other location along flow lumen 412 of CSF drainage catheter, apart from flow control valve.

Sensor 402 is generally any suitable apparatus configured to measure flow through flow lumen 412. As such, sensor 402 may comprise a thermal measuring device, a differential pressure measuring device, a turbine meter, a coriolis, a positive displacement measuring device, an ultrasonic device and/or the like. In one embodiment, sensor 402 generally includes temperature detector 404, such as a thermistor, and a heater. Temperature detector 404 may be positioned either at or on heater or apart from heater. Generally, heater comprises any apparatus suitably configured to heat fluid in the flow path. Flow of CSF through flow lumen 412 and flow path 406 may be monitored as an inverse function of the temperature measured by temperature detector 404, based on a thermal dilution heat transfer model. In one embodiment, heater will be configured to maintain an operating temperature in sensor 402 of between about 35° C. and about 50° C. and preferably between about 35° C. and about 45° C.

Flow path 406 is configured to allow flow of CSF through flow lumen 412, past sensor 404, so that flow rate can be measured. As such, flow path 406 may have any of a variety of suitable configurations. For example, flow path 406 may have a straight cylindrical shape, as illustrated in FIG. 4, or may have an alternate shape, such as straight rectangular, stepped or folded cylindrical or rectangular, or any other suitable configuration. Additionally, flow path 406 may be configured with any of a variety of suitable lengths and diameters. Preferably, flow path 406 will have a diameter that approximates that of the CSF shunt to which it is attached or with which it is coupled. Length of flow path 406 will be chosen based on the accuracy, size, and ease of manufacture of flow monitoring apparatus 400. In one embodiment, flow path 406 will be designed with a length of between about 0.5 cm and about 4 cm and preferably less than about 1 cm. In another embodiment, flow path 406 will be designed to confer fluid resistance of between about 1 cm H₂O/(ml/day) and 20 cm H₂O/(ml/day) and preferably less than about 11 cm H₂O/(ml/day).

According to another aspect of the present invention, transmitter/receiver 408 may include either a transmitter, a receiver, or both. Transmitter/receiver 408 may comprise any suitable device for sending and/or receiving signals. In various embodiments, transmitter/receiver 408 may send and/or receive radio frequency signals, optical signals, ultrasound signals, EMI signals and the like. For example, transmitter/receiver 408 may be configured for receiving an externally generated energy signal, such as a radio frequency signal. Transmitter/receiver 408 may be further configured to provide such received energy to sensor 402. Transmitter/receiver 408 may also be configured to send signals to an external apparatus, for example signals related to measurements by sensor 402.

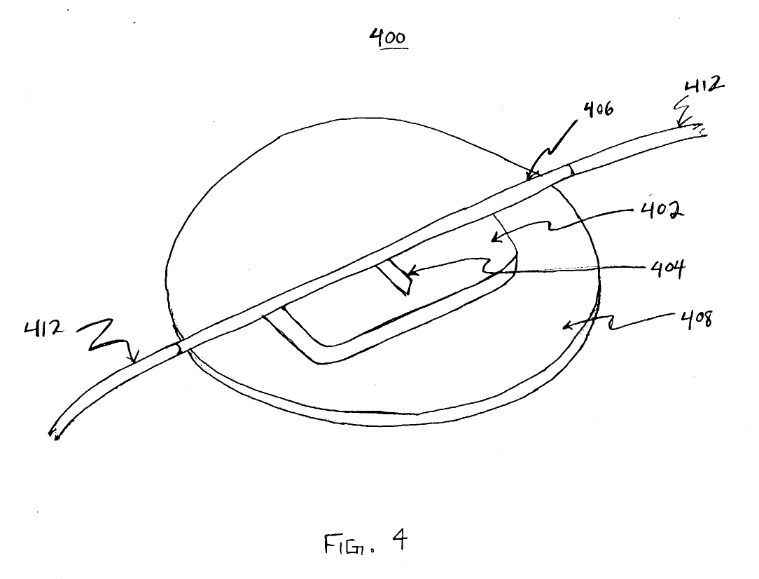

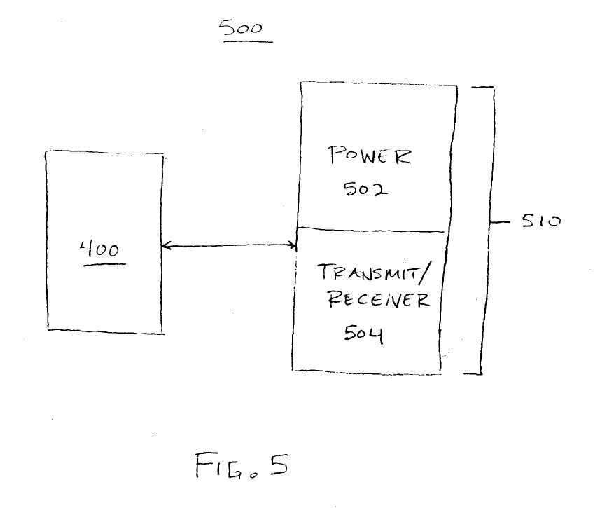

In one embodiment of the present invention, flow monitoring apparatus 400 may be included in a flow monitoring system 500 for draining CSF and monitoring such drainage. An example of such a system is illustrated in FIG. 5. In the illustrated embodiment, flow monitoring system 500 generally includes flow monitoring apparatus 400 and an external apparatus 510. External apparatus 510 includes a power supply 502 and an external transmitter/receiver 504. External transmitter/receiver 504 may transmit signals, receive signals, or both, and is generally configured to communicate with transmitter/receiver 408 of flow monitoring apparatus (FIG. 4). Power supply 502 is generally configured to provide power to flow monitoring apparatus 400. It should be emphasized that flow monitoring system 500 of the present invention contemplates embodiments in which a CSF drainage catheter is included as well as embodiments that do not include a CSF drainage catheter. In embodiments that do not include a CSF drainage catheter, flow monitoring unit 500 will be attachable or otherwise adapted for incorporation into a CSF drainage catheter.

External apparatus 510 may include fewer or additional components and any suitable configuration of external apparatus 510 is contemplated within the scope of the present invention. For example, functions of external transmitter/receiver 504 may be segregated into two or more components, such as an antenna for transmitting signals and a receiver for receiving signals. In another embodiment, external apparatus 510 may include such components as a signal decoder for analyzing signals from flow monitoring apparatus 400, a user interface for enabling a user to receive and input information to external apparatus 510, and/or the like. Furthermore, external apparatus 510 may include separate components which are coupled through any suitable means. For example, external transmitter/receiver 504 may be a separate component that is coupled to the other components in external apparatus 510 via an electrical cable or by other means. Alternatively, external transmitter/receiver 504 may be unconnected from other components of external apparatus 510 and may communicate with one or more of those other components via radio frequency signals, optical signals, ultrasound signals and/or the like. The ability to separate external transmitter/receiver 504 from external apparatus 510 may facilitate placement of external transmitter/receiver 504 at a convenient location to receive signals from flow monitoring apparatus 400.

As is apparent from the foregoing description, flow monitoring apparatus 400 and flow monitoring system 500 may assume any of a variety of configurations without departing from the scope of the present invention. Similarly, many various methods may be contemplated for using the various embodiments of the present invention to monitor CSF flow through a shunt. Thus, the following description of one exemplary method for using apparatus and systems for monitoring CSF flow is provided for descriptive purposes only and is not meant to limit the scope of the invention as described in the claims.

In one embodiment, external apparatus 510 may be used to transmit an interrogation signal to flow monitoring apparatus 400. This interrogation signal may originate, for example, from a user interface and may be transmitted via external antenna 504 to antenna 408. An interrogation signal may include instructions to activate sensor 402, instructions for sensor 402 to heat temperature detector 404, instructions for sensor 402 to heat flow path 408 and/or the like. An interrogation signal may also include the transmission of energy, such as radio frequency energy, to flow monitoring apparatus 400. In response to one or more interrogation signals from external apparatus 510, flow monitoring unit 400 will take at least one measurement which represents flow of CSF through flow lumen 412 and/or flow path 406. For example, in one embodiment where sensor 402 comprises a thermistor or similar thermal measuring device, sensor 402 may heat temperature detector 404. Higher rates of CSF flow through flow path 406 will cause temperature detector 404 to heat at slower rates, while lower rates of CSF flow will allow temperature detector 404 to heat at faster rates.

While or after flow monitoring unit 400 takes one or more measurements, it transmits one or more signals representing those measurements to external apparatus 510 and external apparatus receives those signals and converts them into a rate of CSF flow through flow lumen 412. Any suitable user interface incorporated with external apparatus 510 then allows a user to read the CSF flow rate.

The following example of one embodiment of systems and methods of the present invention is again provided for descriptive purposes only and does not limit the scope of the invention as set forth in the appended claims:

### Example 1

A P₂OBA 103 M Thermoprobe (Thermometrics www.thermometrics.com), measuring 0.5 mm long and 0.5 mm in diameter, was incorporated into a standard peritoneal catheter of 1.1 mm inner diameter. Fluid flow, controlled by maintaining a constant hydrostatic head across a fluid resistor consisting of a small-bore (approximately 0.15 mm inner diameter) tube, was monitored in real time by weighing the accumulated outflow from the system on a Setra digital scale with 1-milligram resolution. The thermistor was configured as a half bridge and was powered using a 1 to 8 volt square wave. This corresponded to mean input powers of approximately 1 mW and 0.1 mW. In this way, the thermistor could be monitored while experiencing minimal self-heating (0.1 mW input), which allowed for measurement of ambient fluid temperature, and in a self-heating mode (1 mW input) which allowed for measurement of thermal dissipation and, hence, fluid flow.

A personal computer, configured with a National Instruments (www.natinst.com) NI 6011E PCI-MIO-16XE-50, was used to source and monitor the sensor—the scale was monitored over a serial port. The system was controlled and monitored by a Microsoft Visual Basic™ program. Sensor performance was then monitored over a number of flows between 0.01 ml/day and 0.09 ml/day. While evaluating performance at any given flow, a no-flow performance was also acquired by intermittently closing the valve at the distal end of the system.

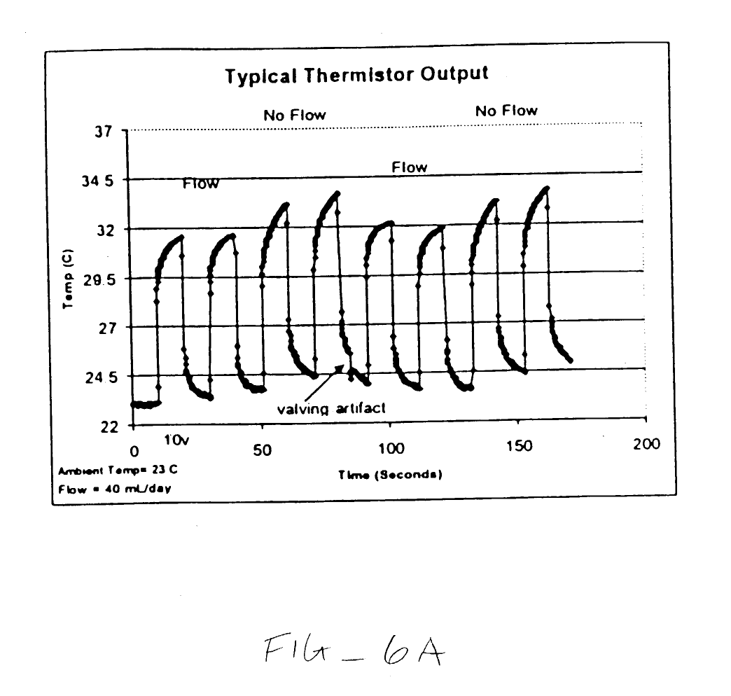

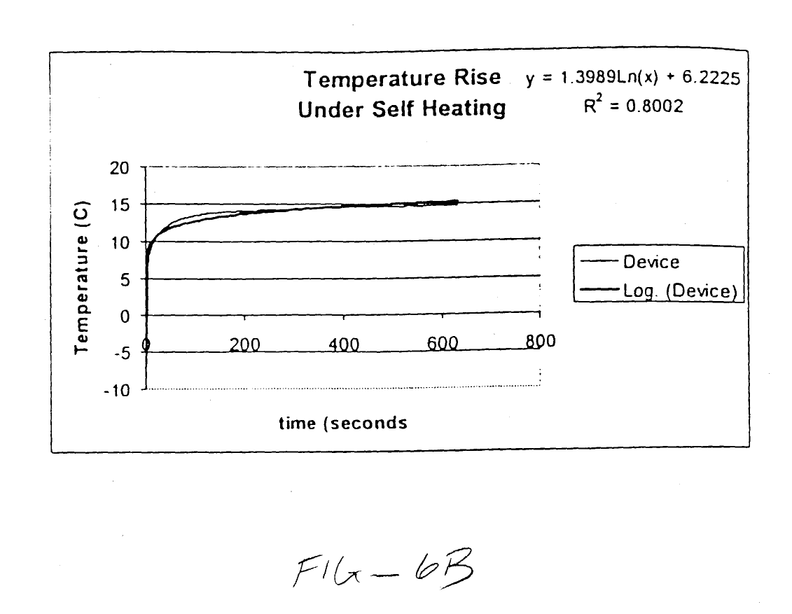

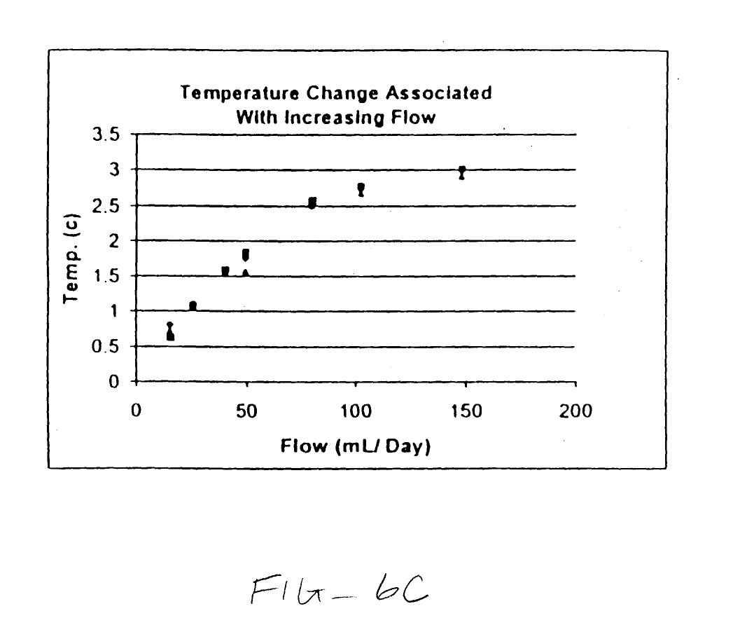

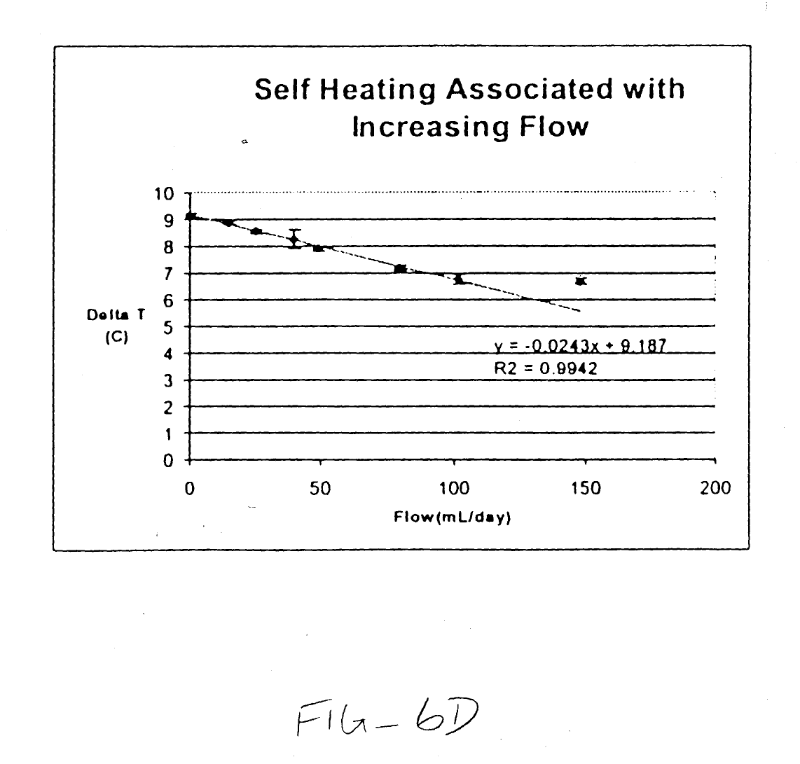

A typical sensor output converted to temperature is presented in FIG. 6a. As shown, the rate of self-heating, following the increase in input voltage, is greater in the no-flow situation than in the flow situation. This difference is significant and can be seen as both a difference in rate of change in temperature and a difference in end point temperature. The difference in end point temperature is on the order of a few degrees at this relatively low flow of 0.028 ml/day. Additionally, given the fixed 10-second duration for the high voltage and low voltage inputs, the thermistor is far from equilibrium temperature in both the flow and no-flow situations. At the end of the 10-second high voltage period, the end point attained in the flow situation is closer to equilibrium than that for the no-flow situation.

FIG. 6b illustrates a compilation of the end point temperature differences observed between flow and no-flow for a range of 7 flows between 0.01 and 0.10 ml/hr. FIG. 6c illustrates the thermistor self-heating temperature rise associated with the increase from 1 to 8 volts as a function of flow rate. Both FIG. 6b and FIG. 6c show a-reduction in sensitivity of the system as flow rate increases. FIG. 6d illustrates self-heating dependent temperature rise as a function of time over an extended period of time.

An implantable system 600 constructed in accordance with the principles of the present invention generally comprises a flow sensor 602 that transfers flow data to signal conditioning circuitry 604 which can convert the measured fluid flow to an electrical signal which is fed to impedance switch (Z switch) 606 which can modify the impedance of an antenna 608 which is attached to the circuitry through a magnetic resonance imaging (MRI) device 608. Optionally, the system 600 may include a level sense circuit that could, for example, request measurements or control other internal functions of the system. Power supply is fed directly by the antenna 602 and will generally have little or no power storage capability. In this way, the sense flow information can be “transmitted” externally with the impedance changes in the antenna.

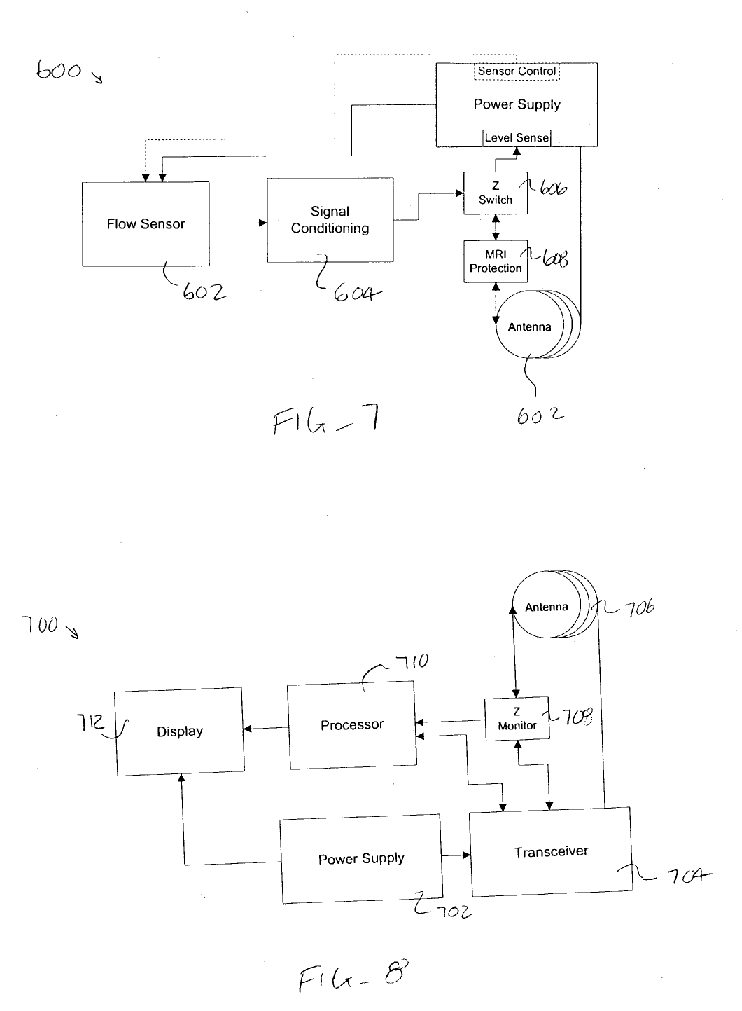

An external transceiver 700 which is suitable to communicate with the implanted system 600 is illustrated in FIG. 8. The system 700 provides power, data and signal processing, and display for the flow measurements obtained from the implanted system 600. The external transceiver 700 can transfer energy from power supply 702 through transceiver unit 704 and antenna 706, typically by placing the antenna proximate the known location of the implanted antenna 602 in system 600. An impedance (Z) monitor 708 attached to the antenna 706 can monitor the impedance displayed by implanted antenna 602 and extract flow rate data that has been transferred into the implanted antenna. Processor 710 can convert the detected impedance data from monitor 708 into numerical data which can then be transferred to a conventional display 712. A user, by observing the displayed flow rate, can then determine the operational status of the implanted shunt.

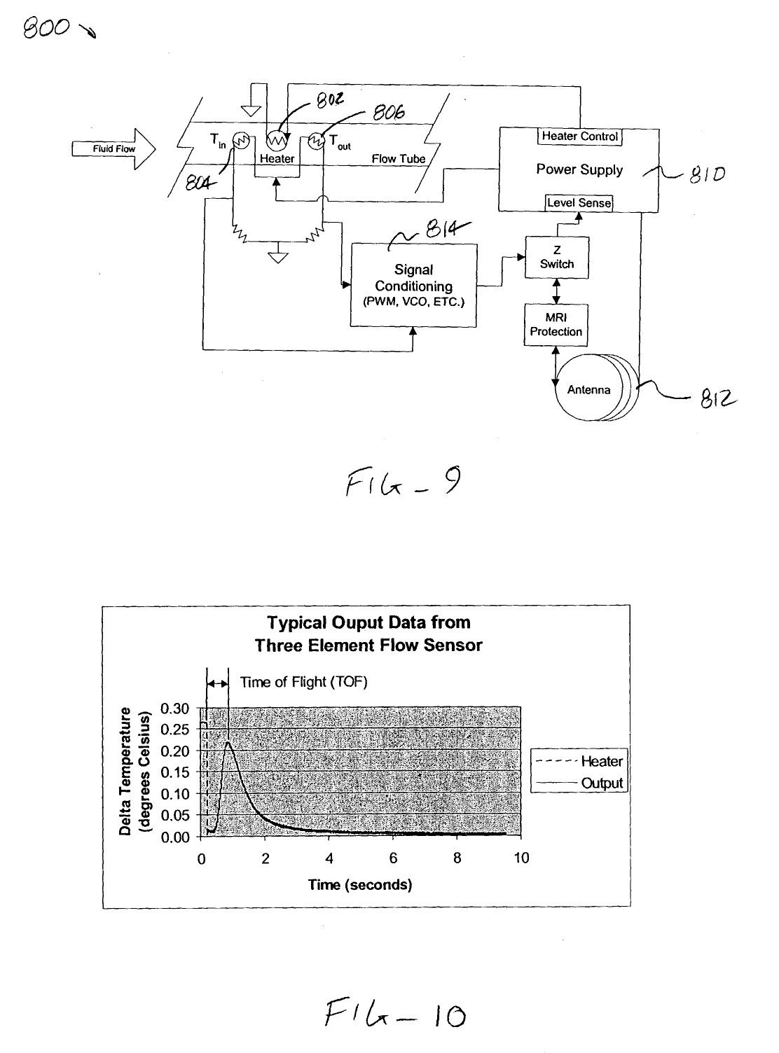

An alternative implantable flow detection system 800 is illustrated in FIG. 9. A system 800 is intended in particular to detect flow through a thermal detection system comprising a heater 802, upstream and downstream temperature sensors 804 and 806, respectively, and a power supply 810. The power supply is powered through radiofrequency transmissions received by antenna 812, and the temperature sensors 804 and 806 are included in a bridge circuit attached to signal conditioning circuitry 814. The power supply controls the heater 802 with a known amount of energy input. By measuring the upstream and downstream temperatures 804 and 806, respectively, and recording the temperature rise, the flow rate of fluid through the flow tube of the implantable shunt can be determined. In particular, as the temperature sensors 804 and 806 are attached in a bridge circuit, the output signal will be proportional to the change in temperature. By generating a heat pulse, and imparting a small temperature increase for a brief time, fluid moving through the shunt will transport the heat downstream to temperature sensor 806. By monitoring the rise and fall in temperature detected at the downstream sensor 806, the rate of flow of CSF through the shunt may be calculated, as described below.

The implanted system 800 may utilize the same external system 700, as illustrated in FIG. 8. In operation, the system 700 may send a request for a flow reading to the antenna 812 of system 800. A link to the implanted device is made by initiating a signal at the operating power frequency. Once the link is made, energy required to power all of the implanted sensor electronics in system 800 is extracted from the radiofrequency signal and the transceiver 704 timer is started. The transceiver 704 then waits either for a predetermined time period or for a “ready” signal from the implanted system 800 before recording the impedance changes generated over time by temperature sensor 806, which are transmitted back as impedance changes through antenna 812. After collecting data for a period of time sufficient to capture the rise and fall in temperature, the radio signal is discontinued and the implanted circuitry shuts down.

The transceiver system 700 averages the received data to remove noise and determines the time difference between turning on of the heater and the corresponding downstream temperature rise. For example, the time between the end of the heater pulse and the maximum temperature read at temperature sensor 806 may be used to determine the CSF flow rate. This determined “Time of Flight (TOF)” is illustrated in FIG. 10 and may be used with a stored flow equation to calculate the flow rate associated with the temperature rise. The calculated flow rate may then be displayed, and the transceiver unit may be ready to make additional flow measurements as just described.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A method for monitoring cerebrospinal fluid (CSF) flow in a normal pressure patient having an implanted CSF drainage shunt, said method comprising:
externally receiving an output signal from a flow sensor in a flow lumen of the implanted CSF drainage shunt, said signal being representative of CSF flow through the flow lumen.

2. A method as in claim 1, further comprising transmitting an interrogation signal to the flow sensor prior to receiving the output signal.

3. A method as in claim 2, wherein the transmitting interrogation signal comprises transmitting power to the flow sensor, wherein said power activates the flow sensor to detect flow and generate the output signal.

4. A method as in claim 3, wherein the flow sensor is selected from the group consisting of a thermal device, a dye release device, a differential pressure measuring device, a turbine meter, an angular momentum measuring device, a positive displacement measuring device, and an accumulator.

5. A method as in claim 3, wherein the flow sensor is a thermal device in which interrogation signal induces heat generation in the flow lumen and the flow sensor measures temperature.

6. A method as in claim 5, wherein the flow sensor produces a signal which is an inverse function of temperature based on a thermal dilution heat transfer model.

7. A method as in claim 5, wherein the flow sensor produces a signal which is based on a thermal diffusion heat transfer model.

8. A method as in any of claims 2 to 7, wherein transmitting comprises directing radiofrequency energy to an antenna coupled to the fluid sensor.

9. A method as in claim 7, wherein the radiofrequency energy provides energy to the flow sensor.

10. A method as in claim 1, wherein the expected CSF flow in the lumen is in the range from 12 ml/day to 1200 ml/day.

11. A method as in claim 1, wherein the signal indicates whether there is a flow of 0.5 ml/hour or greater at the time the signal was generated.

12. A method as in claim 1, wherein the signal indicates whether there has been a cumulative flow through the shunt of at least 12 ml/day.

13. A method for monitoring cerebrospinal fluid (CSF) flow in a patient having an implanted CSF drainage shunt, said shunt assembly having a heat source and a temperature sensor proximate the heat source, said method comprising;
directing energy to heat the heat source, wherein an increase in the CSF flow rate reduces the temperature detected by the temperature sensor;
externally receiving an output signal from the temperature sensor; and
determining based on the output signal whether the shunt is draining CSF.

14. A method as in claim 13, wherein directing comprises transmitting energy selected from the group consisting of radiofrequency energy, ultrasonic energy, and optical energy.

15. A method as in claim 13, wherein the output signal comprises energy selected from the group consisting of radiofrequency energy, ultrasonic energy, and optical energy.

16. An implantable apparatus for draining cerebrospinal fluid (CSF), said apparatus comprising:
an implantable drainage catheter having one end adapted for implantation in a subarachnoid space (SAS), a drainage end adapted for implantation in a drainage space, and a flow lumen therebetween; and
a flow sensor which is coupled to sense flow through the flow lumen of the drainage catheter and which transmits a signal representative of flow through the flow lumen, wherein the sensor is capable of detecting flows at least as low as 12 ml/day.

17. An apparatus as in claim 16, wherein the sensor is adapted to detect a minimum instantaneous flow rate corresponding to 12 ml/day.

18. An apparatus as in claim 16, wherein the sensor is adapted to detect a minimum cumulative flow rate corresponding to 12 ml/day.

19. An implantable apparatus as in claim 16, wherein the implantable drainage catheter includes a first conduit implantable in the SAS, a second conduit implantable in the drainage space, and a flow control valve assembly therebetween.

20. An apparatus as in claim 19, wherein the flow sensor is disposed in or on the flow control valve assembly.

21. An apparatus as in any of claims 16 to 20, wherein the control valve assembly is configured to allow flow rates from 0.01 ml/min to 0.2 ml/min so long as pressure across the valve is in the range from 5 mmH₂O to 450 mm H₂O.

22. An apparatus as in any of claims 16 to 20, wherein the flow sensor is selected from the group consisting of a thermal device, a dye release device, a differential pressure measuring device, a turbine meter, an angular momentum measuring device, a positive displacement measuring device, and an accumulator.

23. An apparatus as in claim 22, wherein the flow sensor comprises a heater and a temperature detector, wherein the heater can be externally energized and the temperature detector can be externally interrogated.

24. An apparatus as in claim 23, wherein the temperature detector is spaced-apart from the heater in a direction toward the drainage end, whereby the flow signal is a function of the temperature measured by the temperature detector based on a thermal dilution heat transfer model.

25. An apparatus as in claim 24, wherein the temperature detector is disposed at or on the heater, whereby the flow signal is a function of the temperature detector based on a thermal diffusion heat transfer model.

26. An implantable apparatus as in any of claims 16 to 20, wherein the flow sensor includes a receiver for receiving an externally generated signal.

27. An implantable apparatus as in claim 26, wherein the receiver receives externally generated energy, and provides energy to the flow sensor.

28. A system for draining cerebrospinal fluid (CSF) and monitoring such drainage, said system comprising;
an implantable drainage catheter having a flow sensor adapted to detect a flow corresponding to a flow rate at least as low as 12 ml/day;
a power supply having an antenna adapted to externally deliver energy to the flow sensor when the flow sensor is implanted; and
an external receiver adapted to receive signals from the flow sensor when the flow sensor is implanted.
